(19) United States
(12) Patent Application Publication
(10) Pub. No.: US 2012/0199139 A1
(43) Pub. Date: Aug. 9, 2012

(54) INTRA-ORAL SALIVA REMOVAL CONTINUOUS POSITIVE AIR PRESSURE DEVICE AND METHOD

(76) Inventors: Eran Lavi, Tel Aviv (IL); David Madjar, Tel Aviv (IL)

(21) Appl. No.: 13/497,072

(22) PCT Filed: Sep. 15, 2010
(86) PCT No.: PCT/IL10/00752

§ 371 Date: Mar. 20, 2012

(30) Foreign Application Priority Data
Sep. 24, 2009 (IL) 201154

(51) Int. Cl. A61C 17/10; A61M 16/00

(52) U.S. Cl. 128/859

(57) ABSTRACT

There is provided an intra-oral saliva-removal device for introduction into a buccal vestibulum between teeth and inner parts of lips and cheeks in a mouth of a patient, the device comprising at least one uni-directional valve adapted to transfer fluids from a buccal side of the device to a lingual side thereof.

### FIELD OF THE INVENTION

The present invention relates generally to devices and methods for providing [symbol] ses intra-orally, and more specifically to ergonomic apparatus and methods for [symbol] liva removal during continuous positive air pressure provision to a mammalian [symbol] bject.

### BACKGROUND OF THE INVENTION

Mammalian subjects require a semi-continuous supply of air, such that the [symbol] ygen level in the brain is retained above a threshold level. There are many [symbol] nditions and situations under which the air supply is temporarily stopped or [symbol] duced. These may include, but are not limited to, sleep apnea, heart attack, epileptic [symbol] izure and drowning. If the subject does not receive oxygen within a number of [symbol] conds/minutes, the result can lead to irreversible brain damage, and, in some cases, [symbol] ath.

Many devices and methods have been developed to ensure a continuous air [symbol] pply to human subjects, such as sleep apnea devices. However, many of the devices [symbol] e cumbersome, uncomfortable and lead to patient non-compliance. Other devices [symbol] e not adapted to deal with patient movement.

The amount of saliva produced by a healthy person per day is estimated to be [symbol] the range of 0.75 liters per day to 1.5 liters per day. This suggests that the amount [symbol] oduced varies from person to person. It is generally accepted though that while [symbol] eeping the amount usually drops significantly, in some cases down to almost zero. A [symbol] reign object, such as a continuous positive airway pressure CPAP device, in the [symbol] ccal sulcus may also induce increased salivation, especially at the beginning of [symbol] tra-oral device use.

A significant reduction of saliva secretion will result after prolonged use, but [symbol] vertheless, a small but annoying amount of saliva may drool between the lips to the [symbol] rner of the mouth. Although this problem has been reported for only a small [symbol] umber of patients, where it exists, it may cause annoyance and inconvenience to the [symbol] tient.

Some publications relating to sleep apnea devices include: U.S. Pat. No. 4,305,387, US [symbol] tent Publication Nos. US2002005201, US2003075182, US2003183227, [symbol] S2003089371, US2005236003, US2007131229A, World Patent Publication Nos. [symbol] O06079149A and W008041237A.

Despite the advances of the inventions described hereinabove, there is still a [symbol] ed to provide CPAP devices and methods which are less cumbersome and of better [symbol] gonomics, thereby providing devices which lend themselves to greater patient [symbol] mpliance. Additionally, there is an unmet need for these devices to prevent saliva [symbol] ooling during wake hours and during sleep.

### SUMMARY OF THE INVENTION

It is an object of some aspects of the present invention to provide intra-oral [symbol] vices and methods for enabling swallowing of saliva when the device is in situ in [symbol] e patient's mouth, concomitantly with providing air to the patient, thereby [symbol] eventing death and injury due to a temporary stoppage of their natural breathing [symbol] ocess.

It is an object of some aspects of the present invention to provide intra-oral [symbol] ep apnea devices and methods for enabling swallowing of saliva when the device is [symbol] situ in the patient's mouth, concomitantly with providing air to the patient, thereby [symbol] eventing death and injury due to a temporary stoppage of their natural breathing [symbol] ocess.

It is an object of some aspects of the present invention to provide saliva [symbol] moval intra-oral devices and methods for providing air to patients thereby [symbol] eventing their death and injury due to a reduction of air intake during their natural [symbol] eathing process. By “removal” is meant transfer from a place within the mouth from [symbol] ich the saliva cannot be easily swallowed to a place from which it can be [symbol] allowed.

In some embodiments of the present invention, improved ergonomic methods [symbol] d apparatus are provided for preventing death and injury in patients suffering from [symbol] eep apnea, stroke, heart attack, trauma, COPD, Alzheimer and other conditions.

In other some embodiments of the present invention, a method and a saliva [symbol] moval intra-oral interface for providing continuous positive airway pressure [symbol] PAP) to a patient.

In additional some embodiments for the present invention, a saliva-removal [symbol] traoral interface is provided which is non-obtrusive, comfortable, does not impinge [symbol] or touch the soft palate, tongue or teeth. The interface is simple to use, lightweight [symbol] d ergonomically designed.

In additional embodiments for the present invention, an ergonomically formed [symbol] tra-oral interface is provided for providing an unconscious, semi-conscious or [symbol] eeping patient with continuous or semi-continuous saliva removal coupled with [symbol] ntinuous positive airway pressure (CPAP).

In further embodiments of the present invention, an interface provides for [symbol] ntinuous positive airway pressure (CPAP) maintenance in a patient.

In further embodiments of the present invention, an interface provides for non [symbol] vasive ventilation in a patient.

In order to prevent saliva from exiting the oral cavity via the lips and the [symbol] companied patient inconvenience, some embodiments of the present invention are [symbol] rected to methods and apparatus for enabling the accumulated saliva to move along [symbol] normal path. Thus devices of the present invention are constructed and configured [symbol] r placing in between the teeth and gums, yet enabling saliva to flow from the [symbol] sterior buccal sulcus where it is excreted by the parotis gland to a lingual side of the [symbol] eth in the interior of the oral cavity, where the combined action of the tongue and [symbol] e muscles involved in swallowing enable the saliva to be swallowed.

Some of the devices of the present invention prevent the passage of saliva [symbol] om a lingual side to a buccal side thereof.

When a sleeping patient has a device of the present invention in his mouth, [symbol] liva tends to accumulate in front of a posterior bulge of the device due to the device [symbol] orphology. At the locations of saliva accumulation inside the oral cavity, the [symbol] terface is structured in a way that directs the saliva elsewhere. The problem of saliva [symbol] cumulation is solved by positioning one or more unidirectional valves at the site of [symbol] liva accumulation.

The valve is constructed and configured to transfer saliva to the lingual side of [symbol] e device during a normal swallowing cycle, which involves various oral muscle [symbol] ntractions. The valve is preferably opened uni-directionally to allow for saliva [symbol] nsfer from a buccal side to a lingual side thereof.

The valve is activated by normal and regular muscle contraction which takes [symbol] ace during each swallowing cycle. Muscle relaxation after swallowing allows for [symbol] lve closure. Closure of the valve is innate to the elastic property of the material. [symbol] hen the valve is closed, a complete seal of the oral cavity from the outside is [symbol] stored.

Upon a swallowing reflex, the valve is opened for a short duration. Saliva is [symbol] tively forced through the valve to the lingual side because the position of the valve [symbol] front of the posterior bulge allows the contracting muscles to sweep the saliva [symbol] wards the opened orifice of the valve.

The valve is not a device implanted into the SomnuSeal but rather, is an [symbol] tegral part of the SomnuSeal silicone material and so its use is safe and it may not [symbol] ar or depart from the main body of the device.

The valves are integral parts of the device/interface thereby enabling easy [symbol] eaning and hygienic and safe operation thereof.

The valves are designed such that air or saliva cannot pass from the lingual [symbol] de of the valve to the buccal side thereof. These uni-directional valves, also known [symbol] “check valves”, are located in the mouth at areas at which saliva accumulates. [symbol] ese places are either at the anatomical places of saliva accumulation and/or at [symbol] aces where the saliva accumulates as a result of the morphology of the [symbol] vice/interface.

This uni-directional valve allows saliva to move to the inside of the oral cavity [symbol] ing the mechanism of a uni-directional valve with or without the help of muscle [symbol] ntraction during the saliva swallowing, which occurs naturally during such a [symbol] ocess. These uni-directional valves do not allow air or fluids to move from the [symbol] rection of inside the oral cavity to the buccal sulcus.

This valve does not interfere with the oral device/interface sealing. and if it [symbol] es, the interference will be for a very short period of less than 5 seconds (calculated [symbol] cording to normal time span of regular saliva swallowing).

The uni directional valves may be placed in parallel to the muscle contraction [symbol] ectors.

The transfer of the saliva into the oral cavity through the uni-directional valve [symbol] ll occur when enough saliva accumulates near the valve and when the patient [symbol] allows. The swallowing action—contraction of the lips, will exert pressure on the [symbol] cumulated saliva that will have to pass the uni-directional valve as it will be the [symbol] st resistant path for it.

Then, the accumulated saliva that passed the uni-directional path will be [symbol] rected by a duct like structure to the sides of the interface where the saliva will be [symbol] cked by the saliva swallowing process of the patient.

Another solution, two channel like structures between the physical location [symbol] here the saliva accumulates (either naturally or as a result of the interface [symbol] orphology) and the distal part of the buccal sulcus.

This structure allows the saliva to flow freely towards the inner part of the oral [symbol] vity and be swallowed naturally from there on.

There is thus provided according to some embodiments of the present [symbol] vention, an intra-oral saliva-removal device for introduction in the buccal [symbol] stibulum between teeth and inner part of lips and cheeks in a mouth of a patient, the [symbol] vice including at least one uni-directional valve adapted to transfer fluids from a [symbol] ccal side of the device to a lingual side thereof.

Additionally, according to some embodiments of the present invention, the [symbol] vice is a saliva-removal continuous positive air pressure (SRCPAP) device.

Furthermore, according to some embodiments of the present invention, the [symbol] vice is a CPAP device.

Moreover, according to some embodiments of the present invention, the [symbol] vice includes at least two uni-directional valves.

Further, according to some embodiments of the present invention, the device [symbol] cludes at least four uni-directional valves.

Additionally, according to some embodiments of the present invention, the [symbol] vice is constructed and configured to transfer saliva from the buccal side of the [symbol] vice to the lingual side thereof.

According to some embodiments of the present invention, the saliva is adapted [symbol] swallowed on the lingual side.

Additionally, according to some embodiments of the present invention, the at [symbol] ast two uni-directional valves are constructed and configured to prevent backflow of [symbol] ids from the lingual side to the buccal side of the device.

Moreover, according to some embodiments of the present invention, the [symbol] vice may include;

a) a hollowed ellipsoid tube section which opens towards the teeth at a first end;

b) an intra-oral section extending perpendicularly from a second end of the hollowed ellipsoid tube section, the intra-oral section including;
i. a buccal hollowed ellipsoid surface; and
ii. a lingual rim projecting from a circumferential border of the hollowed ellipsoid surface, thereby forming a circumferential hollow lip, wherein the hollow lip is adapted to bulge upon receiving air thereby forming a circumferential air pocket within the circumferential hollow lip.

Further, according to some embodiments of the present invention, the intra- [symbol] al section is adapted to be inserted within buccal sulci occupying substantially the [symbol] tire volume of a buccal sulcus potential space upon receiving air in such a way that [symbol] cilitates oral cavity sealing, and to retain oral cavity sealing in a sealed state without [symbol] cupying the entire volume of a buccal sulcus potential space.

Furthermore, according to some embodiments of the present invention, the at [symbol] ast one valve is adapted to open when cheek muscles contract.

Yet further, according to some embodiments of the present invention, the at [symbol] ast one valve is adapted to close when cheek muscles relax.

Furthermore, according to some embodiments of the present invention, the [symbol] vice weighs less than 60 grams.

Additionally, according to some embodiments of the present invention, the [symbol] vice weighs 20 to 50 grams.

Furthermore, according to some embodiments of the present invention, the [symbol] vice is constructed and configured to conform to mouth physiology of a patient.

Moreover, according to some embodiments of the present invention, the [symbol] vice is constructed and configured to passively form a seal by occupying a potential [symbol] ace between the lips and gums of a user.

Furthermore, according to some embodiments of the present invention, the [symbol] vice is constructed and configured to supply sufficient air to the patient at an air [symbol] essure of 2-25 cm H₂O.

Moreover, according to some embodiments of the present invention, the [symbol] rcumferential hollow lip is adapted to be inflated by exhaled air of the user.

Furthermore, according to some embodiments of the present invention, the [symbol] vice includes a biocompatible polymer.

Additionally, according to some embodiments of the present invention, the [symbol] rcumferential hollow lip includes collapsible portions.

Furthermore, according to some embodiments of the present invention, the [symbol] llapsible portions exhibit a pre-loaded force which is adapted to press gently onto [symbol] e gums and lips of the user upon insertion to a mouth of the user, thereby forming a [symbol] al.

Moreover, according to some embodiments of the present invention, the intra- [symbol] al section is provided with a central part formed with an aperture in communication [symbol] th the tube, and right and left longitudinally extending projections adjoining, and of [symbol] bstantial bilateral symmetry with respect to, the central part, each of the projections [symbol] ving adjoining upper and lower regions and each of the regions having adjoining [symbol] oximal and distal portions, and wherein each of the projections is dimensioned such [symbol] at a distal portion has a thickness substantially equal to, or greater than, a buccal [symbol] lcus potential space gap, and is configured, when inserted within a buccal sulcus, in [symbol] ch a way so as to adhere to the oral mucosa, to occupy substantially the entire [symbol] lume of buccal sulcus potential space, and to seal the oral cavity.

Furthermore, according to some embodiments of the present invention, a distal [symbol] rtion is considerably thicker than an adjoining proximal portion and than a [symbol] rresponding distal portion of the buccal sulcus potential space to such a degree that [symbol] per and lower lip portions disposed buccally to the central part are urged to [symbol] alingly engage the tube; and wherein the distal portion is also wider buco-lingually, [symbol] ereby adapted to form a larger air pocket than that formed at more proximal parts of [symbol] e interface, thereby allowing for a good seal between posterior portions of the [symbol] terface and the gums of the patient.

Yet further, according to some embodiments of the present invention, the [symbol] vice is constructed and configured to supply sufficient air to the patient at an air [symbol] essure of 2-10 cm H₂O.

Additionally, according to some embodiments of the present invention, the [symbol] vice is constructed and configured to supply sufficient air to the patient at an air [symbol] essure of 4-8 cm H₂O.

Furthermore, according to some embodiments of the present invention, the [symbol] vice is constructed and configured to supply sufficient air to the patient at an air [symbol] essure reduced by 10-60% relative to an existing CPAP interface.

Typically, the intra-oral saliva-removal device is constructed and configured [symbol] supply sufficient air to the patient at an air pressure reduced by 15-50% relative to [symbol] existing CPAP interface.

Furthermore, according to some embodiments of the present invention, there is [symbol] ovided a system for continuously providing a user with sufficient air, the system [symbol] cluding;

a) a device as described herein;

b) at least one set of coils disposed in the device;

c) at least one sensor adapted to receive data from a device neighborhood;

d) a flow generator unit adapted to provide air to the device; and

e) a data processing unit constructed and configured to;
i. process and store the data; and
ii. provide signals responsive to the data to at least one of the coils and an air inlet controller.
iii. provide signals responsive to the data or signals from the flow generator unit.

Furthermore, according to some embodiments of the present invention, the [symbol] stem is adapted to prevent at least one of sleep apnea, snoring and hypopnea.

Additionally, according to some embodiments of the present invention, the [symbol] nsor is selected from an integral pulse oximeter and a CO-oximeter.

Furthermore, according to some embodiments of the present invention, a [symbol] ethod is provided for saliva removal during provision of continuous positive air [symbol] essure to a patient, the method including placing an intra-oral saliva-removal device [symbol] described herein in the buccal vestibulum between teeth and inner part of lips and [symbol] eeks in a mouth of the patient thereby enabling the flow of saliva from a buccal side [symbol] the device to the lingual side thereof.

### BRIEF DESCRIPTION OF THE DRAWINGS

The invention will now be described in connection with certain preferred [symbol] bodiments with reference to the following illustrative figures so that it may be [symbol] ore fully understood.

With specific reference now to the figures in detail, it is stressed that the [symbol] rticulars shown are by way of example and for purposes of illustrative discussion of [symbol] e preferred embodiments of the present invention only and are presented in the [symbol] use of providing what is believed to be the most useful and readily understood [symbol] scription of the principles and conceptual aspects of the invention. In this regard, [symbol] attempt is made to show structural details of the invention in more detail than is [symbol] cessary for a fundamental understanding of the invention, the description taken [symbol] ith the drawings making apparent to those skilled in the art how the several forms of [symbol] e invention may be embodied in practice.

In the drawings:

FIG. 1 is a simplified pictorial illustration of a front view of a SRCPAP (saliva- [symbol] moval continuous positive air pressure) intra oral device, in accordance with an [symbol] bodiment of the present invention;

FIG. 2A is a simplified pictorial illustration of a side view of the SRCPAP [symbol] vice of FIG. 1, in accordance with an embodiment of the present invention;

FIG. 2B is a simplified pictorial illustration of a cross section of a buccal [symbol] rtion along A-A of the SRCPAP device of FIG. 1, in accordance with an [symbol] bodiment of the present invention;

FIG. 3A is a simplified pictorial illustration of a side view of vertical cross [symbol] ction of the SRCPAP device of FIG. 1 with a valve in a closed position, in [symbol] cordance with an embodiment of the present invention; and

FIG. 3B is a simplified pictorial illustration of a side view of vertical cross [symbol] ction of the SRCPAP device of FIG. 1 with a valve in an open position, in [symbol] cordance with an embodiment of the present invention; and

In all the figures similar reference numerals identify similar parts.

### DETAILED DESCRIPTION OF PREFERRED EMBODIMENTS

In the detailed description, numerous specific details are set forth in order to [symbol] ovide a thorough understanding of the invention. However, it will be understood [symbol] those skilled in the art that these are specific embodiments and that the present [symbol] vention may be practiced also in different ways that embody the characterizing [symbol] atures of the invention as described and claimed herein.

All terms used herein are in accordance with the definitions and teachings of [symbol] orld Patent Publication No. WO08041237, incorporated herein by reference. '237 [symbol] aches a shield, having a buccal portion substantially equal to, or greater than, a [symbol] ccal sulcus potential space gap. Additionally, Israel Patent Application No. 197330 DISCOVER MEDICAL DEVICES LTD., teaches intra-oral self adaptable [symbol] ntinuous positive airway pressure (CPAP) interface and method.

The present invention provides intra-oral interfaces for providing continuous [symbol] sitive airway pressure (CPAP) to a patient. Various designs of the hollowed [symbol] terfaces, adapted to create air pockets within the interfaces are described herein, but [symbol] ould not be deemed as limiting.

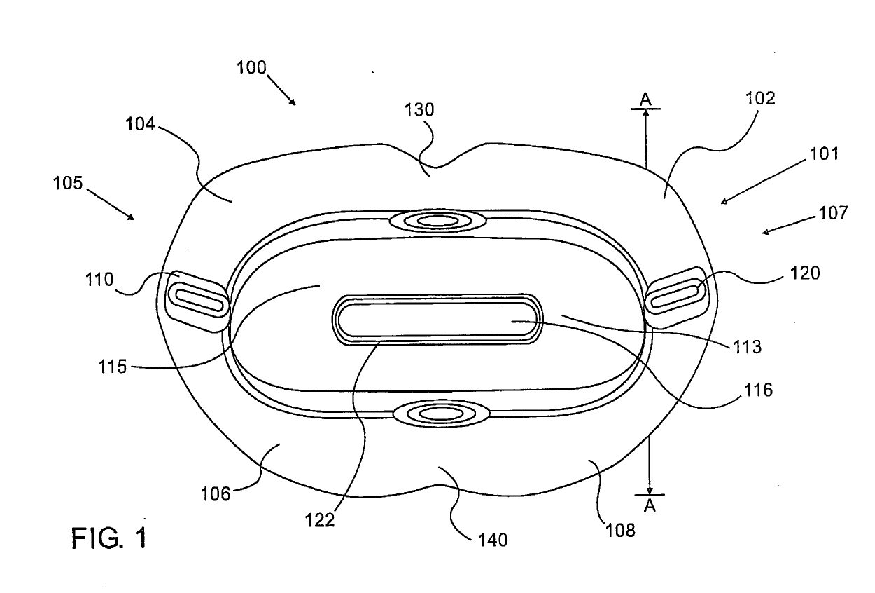

Reference is now made to FIG. 1, which is a simplified pictorial illustration of [symbol] front view of a SRCPAP (saliva-removal continuous positive air pressure) intra oral [symbol] vice 100, in accordance with an embodiment of the present invention.

Device 100 comprises an intraoral hollowed ellipsoid tube section 122, and an [symbol] ra-oral section 101, which serve as a barrier between the oral cavity and [symbol] ospheric pressure air. Section 101, in communication with the source of positive [symbol] pressure has left and right arcuate projections 105 and 107 of bilateral symmetry [symbol] th respect to tube section 122. Projections 101 and 103 are configured to match the [symbol] ternal anatomy of a patient's mouth. Device 100 comprises at least one uni- [symbol] rectional valve 110, 120, 130, 140.

Tube section 122 is constructed and configured as a male portion to fit onto a [symbol] male portion (not shown, but described in IL197330) of an adapter element for [symbol] nnecting to an extra-oral gas supply. The tube section is made out of a [symbol] compatible polymer and is shaped to conform to the mouth opening during rest. It [symbol] of a generally elliptical cross-section with flattened horizontal upper and lower [symbol] des.

Device 100 allows CPAP air or other gases to be delivered through tube [symbol] ction 122. Exhaled gases are discharged from the tube section (and in a small [symbol] mber of cases, some of it may exit through the patient's nostrils).

According to some embodiments, the patient is also provided with nostril [symbol] ngs or stoppers to prevent exhaled air from escaping via the nostrils.

Some of the problems associated with prior art interfaces includes that they are [symbol] avy, cumbersome and are generally uncomfortable. In sharp contrast, the [symbol] terfaces/devices of the present invention are:

a) much lighter, thinner in vertical cross-section and do not significantly weigh down on the gums and lips within the mouth, in comparison with the interfaces of WO08041237;

b) comprise a lingual rim which is constructed and configured to form circumferential hollow lip of the interface, wherein the hollow lip is adapted to bulge upon receiving exhaled and existing air, thereby forming a circumferential air pocket within the circumferential hollow lip;

c) do not press down on any part of the tongue, does not touch the tongue or soft palate and does not protrude into the oral cavity lingual to the teeth leading to a hyperactive gag reflex;

d) do not press down on any part of the skin and lips externally to the oral cavity; and

e) Self adaptable to the physical intra oral structure of each user.

The hollowed ellipsoid tube section 122 is connected perpendicularly at an [symbol] tra-oral end to two thin central sections 113, 115, disposed respectively to the left [symbol] d right of a central vertical axis 151. Surrounding the central sections are four [symbol] ccal bulging portions, namely an upper left buccal portion 102, an upper right [symbol] ccal portion 104, a lower right buccal portion 106 and a lower left buccal portion [symbol] 8. At each distal part of the interface, there is a hollow section which acts as a [symbol] llector of air. It comprises thicker silicon lips and occupies the space adjacent to the [symbol] sterior teeth.

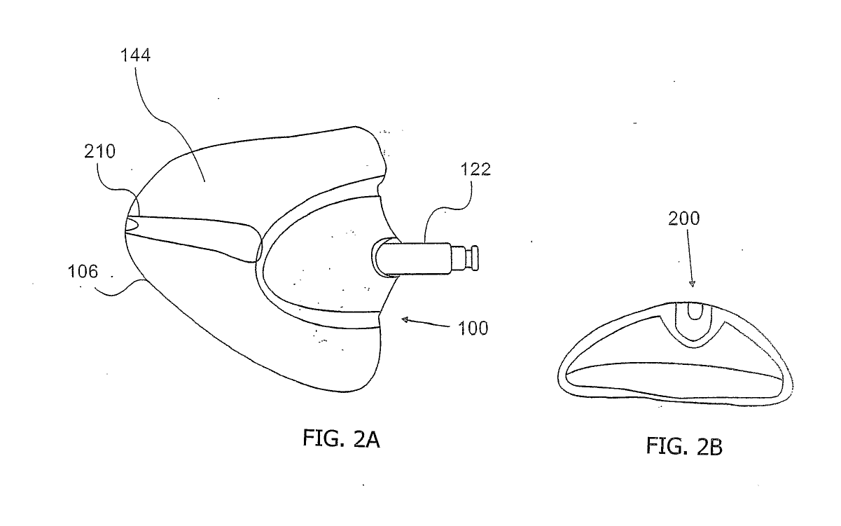

Reference is now made to FIG. 2A, which is a simplified pictorial illustration [symbol] a side view 200 of the SRCPAP device of FIG. 1, in accordance with an [symbol] bodiment of the present invention. Disposed between upper right buccal portion [symbol] 4 and lower right buccal portion 106 is a one way valve 210 which allows passage [symbol] fluid from the buccal side of the device to the lingual side.

Reference is now made to FIG. 2B, which is a simplified pictorial illustration [symbol] a cross section 220 of a buccal portion along A-A of the SRCPAP device of FIG. 1, [symbol] accordance with an embodiment of the present invention.

The valve is constructed and configured to transfer saliva to the lingual side of [symbol] e device during a normal swallowing cycle, which involves various oral muscle [symbol] ntractions. The valve is preferably opened uni-directionally to allow for saliva [symbol] nsfer from a buccal side to a lingual side thereof. It should be understood that there [symbol] ay be a number of valves 210, disposed at various positions on the device, which [symbol] ovide passage of fluids from the buccal side of the device to the lingual side. [symbol] oreover, though the figures show one type of device, this should not be construed as [symbol] iting. The devices of the present invention may be any suitable intra-oral device.

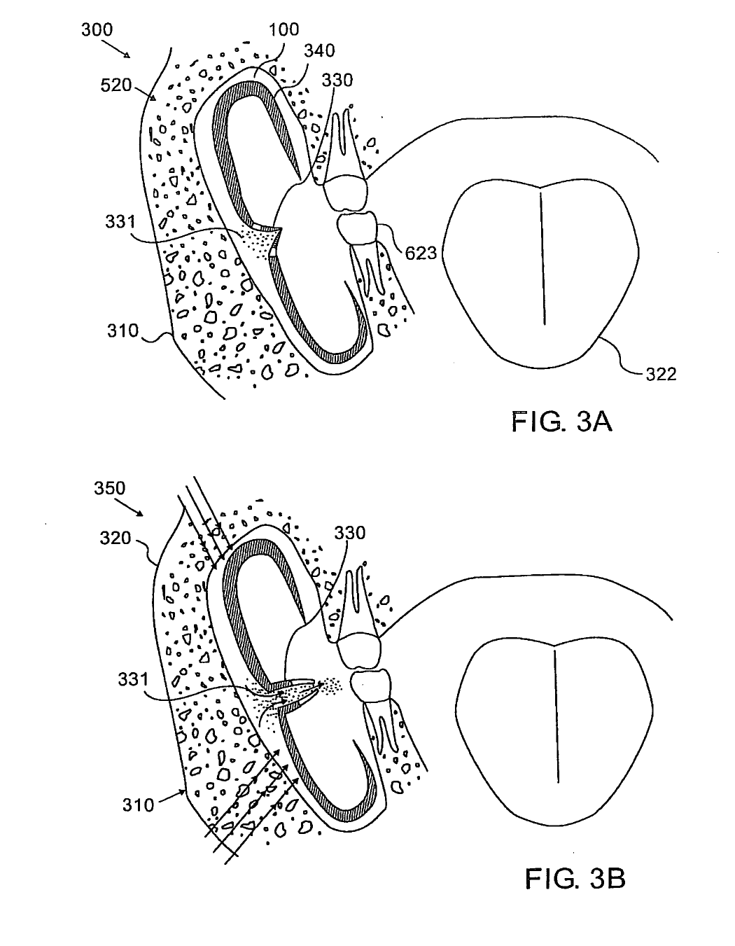

Reference is now made to FIG. 3A, which is a simplified pictorial illustration [symbol] a side view 300 of vertical cross section of the SRCPAP device of FIG. 1 with a [symbol] lve 330 in a closed position when the cheek muscles 310, 320 are relaxed.

Reference is also made to FIG. 3B, which is a simplified pictorial illustration [symbol] a side view 350 of vertical cross section of the SRCPAP device of FIG. 1 with valve [symbol] 0 in an open position, in accordance with an embodiment of the present invention.

This uni-directional valve 330 allows saliva to move to the inside of the oral [symbol] vity using the mechanism of uni-directional valve 330, with or without the help of [symbol] uscle contraction during the saliva swallowing, which occurs naturally during such a [symbol] ocess. These uni-directional valves do not allow air or fluids to move from the [symbol] ection of inside oral cavity 622 to the buccal sulcus.

This valve does not interfere with the oral device/interface sealing. and if it [symbol] es, the interference will be for a very short period of less than 5 seconds (calculated [symbol] cording to normal time span of regular saliva swallowing).

The uni directional valves may be placed in parallel to the muscle contraction [symbol] ctors.

The transfer of the saliva into the oral cavity through the uni directional valve [symbol] ll occur when enough saliva accumulates near the valve and when the patient [symbol] allows. The swallowing action—contraction of the lips, will exert pressure on the [symbol] cumulated saliva that will have to pass the unidirectional valve as it will be the least [symbol] sistant path for it.

Then, the accumulated saliva that passed the uni-directional path will be [symbol] ected by a duct like structure to the sides of the interface where the saliva will be [symbol] cked by the saliva swallowing process of the patient.

In another solution may be applied with two channel-like structures (valves) [symbol] tween the physical location where the saliva accumulates (either naturally or as a [symbol] sult of the interface morphology) and the distal part of the buccal sulcus.

This structure allows the saliva to flow freely towards the inner part of the oral [symbol] vity and be swallowed naturally from there on.

The references cited herein teach many principles that are applicable to the [symbol] esent invention. Therefore the full contents of these publications are incorporated [symbol] reference herein where appropriate for teachings of additional or alternative [symbol] tails, features and/or technical background.

It is to be understood that the invention is not limited in its application to the [symbol] tails set forth in the description contained herein or illustrated in the drawings. The [symbol] vention is capable of other embodiments and of being practiced and carried out in [symbol] rious ways. Those skilled in the art will readily appreciate that various) [symbol] odifications and changes can be applied to the embodiments of the invention as [symbol] reinbefore described without departing from its scope, defined in and by the [symbol] pended claims.

1-36. (canceled)

37. An intra-oral saliva-removal device for continuous positive air pressure (CPAP) provision to a mammalian subject, the device being adapted for introduction into a buccal vestibulum between teeth and inner parts of lips and cheeks in a mouth of the subject, the device comprising at least one uni-directional valve adapted to transfer fluids from a buccal side of the device to a lingual side thereof, and wherein said at least one valve is adapted to open when cheek muscles contract during normal swallowing.

38. An intra-oral saliva-removal device according to claim 37, wherein said at least one valve is adapted to close when cheek muscles relax after swallowing is over.

39. An intra-oral saliva-removal device according to claim 38, wherein said at least one valve is adapted to prevent air or fluids to move from the direction from an inside oral cavity to a buccal sulcus.

40. An intra-oral saliva-removal device according to claim 37, wherein said device weighs 20 to 50 grams.

41. An intra-oral saliva-removal device according to claim 37, wherein said device is constructed and configured to supply sufficient air to the subject at an air pressure reduced by 15-50% relative to an existing CPAP device.

42. An intra-oral saliva-removal device according to claim 37, wherein said device is constructed and configured to supply sufficient air to the subject at an air pressure of 2-25 cm H₂O.

43. An intra-oral saliva-removal device according to claim 37, wherein the device is a saliva-removal continuous positive air pressure (SRCPAP) device.

44. An intra-oral saliva-removal device according to claim 37, wherein said at least one uni-directional valve is disposed in front of a posterior bulge, thereby allowing contracting muscles to sweep saliva towards an opened orifice of the valve.

45. An intra-oral saliva-removal device according to claim 37, wherein said at least one valve is of a duct-like structure, adapted to enable saliva to be swallowed on a lingual side in a regular way.

46. An intra-oral saliva-removal device according to claim 37, wherein said device is constructed and configured to conform to mouth physiology of the subject.

47. An intra-oral saliva-removal device according to claim 37, comprising at least two uni-directional valves each disposed at a site of saliva accumulation, said valves being channel-like structures constructed and configured to prevent backflow of fluids from the lingual side to the buccal side of the device.

48. An intra-oral saliva-removal device according to claim 47, wherein the device comprises at least four uni-directional valves.

49. An intra-oral saliva-removal device according to claim 47, wherein said at least two uni-directional valves are channel-like structures, configured to be placed in parallel to muscle contraction vectors.

50. An intra-oral saliva-removal device according to claim 37, the device comprising:
a hollowed ellipsoid tube section which opens towards the teeth at a first end; and
an intra-oral section extending perpendicularly from a second end of said hollowed ellipsoid tube section, the intra-oral section comprising:
i. a buccal hollowed ellipsoid surface; and
ii. a lingual rim projecting from a circumferential border of said hollowed ellipsoid surface, thereby forming a circumferential hollow lip, wherein said hollow lip is adapted to bulge upon receiving air thereby forming a circumferential air pocket within the circumferential hollow lip.

51. An intra-oral saliva-removal device according to claim 50, wherein said intra-oral section is adapted to be inserted within buccal sulci occupying substantially the entire volume of a buccal sulcus potential space by occupying a potential space between the lips and gums of said subject and wherein said device is adapted, upon receiving air, to facilitate oral cavity sealing, and to retain oral cavity sealing in a sealed state.

52. An intra-oral saliva-removal device according to claim 50, wherein said circumferential hollow lip is adapted to be inflated by exhaled air of the subject.

53. An intra-oral saliva-removal device according to claim 50, wherein the circumferential hollow lip comprises collapsible portions.

54. An intra-oral saliva-removal device according to claim 37, wherein said device is adapted to provide non-invasive ventilation to said subject.

55. An intra-oral saliva-removal device according to claim 42, wherein said device is constructed and configured to supply sufficient air to the subject at an air pressure of 4-8 cm H₂O.

56. An intra-oral saliva-removal device according to claim 55, wherein said device is constructed and configured to supply sufficient air to the subject at an air pressure reduced by 10-60% relative to an existing CPAP interface.
